(19) United States
(12) Patent Application Publication
(10) Pub. No.: US 2011/0152579 A1
(43) Pub. Date: Jun. 23, 2011

(54) METHOD FOR THE HYDROXYLATION OF PHENOL

(76) Inventor: Laurent Garel, Lyon (FR)

(21) Appl. No.: 12/996,866

(22) PCT Filed: Jun. 8, 2009
(86) PCT No.: PCT/EP09/57030

§ 371 Date: Mar. 7, 2011

(30) Foreign Application Priority Data
Jun. 9, 2008 (FR) 08 03194

(51) Int. Cl. C07C 37/08

(52) U.S. Cl. 568/768

(57) ABSTRACT

The subject of the present invention is a method for the hydroxylation of phenol by hydrogen peroxide. The method of the invention for the hydroxylation of phenol to pyrocatechol and hydroquinone in a pyrocatechol/hydroquinone ratio between 1.7 and 2.3, by reaction of the phenol with hydrogen peroxide, in the presence of a catalyst, is characterized by the fact that the reaction is carried out in the presence of an effective amount of a hydroxyaromatic sulfonic acid.

A subject-matter of the present invention is a process for the hydroxylation of phenol with hydrogen peroxide.

A search is currently underway for a process for the hydroxylation of phenol to give hydroquinone (HQ) and pyrocatechol (PC) which results predominantly in pyrocatechol.

It turns out that, in order to meet the market demand, it is important to have available an industrial process which makes it possible to increase the production of pyrocatechol formed with respect to the amount of hydroquinone.

Numerous processes for the hydroxylation of phenols are described in the prior art.

Mention may be made, inter alia, of patent FR-A 2 071 464, which relates to a very important industrial process for the hydroxylation of phenols and phenol ethers which makes it possible to access in particular hydroquinone and pyrocatechol when this process is applied to phenol.

Said process consists in carrying out the hydroxylation, with hydrogen peroxide, in the presence of a strong acid. Among these strong acids, sulfuric acid, p-toluenesulfonic acid and perchloric acid are the most commonly used.

Although this process is highly advantageous, it exhibits the disadvantage of requiring, at low temperature, the use of a significant amount of catalyst ranging up to 20% of the weight of hydrogen peroxide employed. In the case of a lower amount, a greater reaction time is required, for example 10 hours.

Furthermore, FR-A 2 266 683 has described a process which consists in carrying out the hydroxylation of phenol in the presence of a ketone. This results in an improvement in the yield of the reaction to give hydroquinone and pyrocatechol. All the examples described result in a greater amount of pyrocatechol than that of hydroquinone and the PC/HQ ratio varies only between 1.19 and 1.73.

EP-A 0 480 800 has provided, on the other hand, a process which makes it possible to increase the amount of hydroquinone formed with respect to the amount of pyrocatechol by employing a ketone of aromatic type.

In accordance with the process described in EP-A 0 480 800, the presence of this type of ketone during the hydroxylation of phenol acts on the regioselectivity of the reaction and PC/HQ ratios between 0.9 and 1.1 are advantageously obtained.

One of the objects of the invention is to provide a process for the hydroxylation of phenol which makes it possible to obtain a greater amount of pyrocatechol than the amount of hydroquinone.

Another object of the invention is to provide a process for the hydroxylation of phenol which makes it possible to obtain a pyrocatechol/hydroquinone ratio of between 1.7 and 2.3 (limits included) and preferably between 1.9 and 2.2.

Another object of the invention is to provide a process for the hydroxylation of phenol which makes it possible to obtain more pyrocatechol while retaining high yields of diphenols.

More particularly, a subject matter of the present invention is a process for the hydroxylation of phenol to give pyrocatechol and hydroquinone in a pyrocatechol/hydroquinone ratio of between 1.7 and 2.3 by reaction of phenol with hydrogen peroxide in the presence of a catalyst, characterized in that the reaction is carried out in the presence of an effective amount of a hydroxyaromaticsulfonic acid corresponding to the following formula:

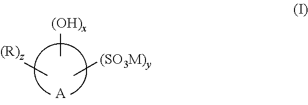

in said formula:
A symbolizing a benzene or naphthalene ring, it being possible for said ring to carry one or more identical or different substituents R,
M representing a hydrogen atom and/or a cation of a metal element from Group Ia of the Periodic Table or an ammonium cation,
x being equal to 1, 2 or 3, preferably 1 or 2,
y being equal to 1 or 2,
z being a number from 0 to 4, preferably 0, 1 or 2.

A preferred embodiment of the invention is a process for the hydroxylation of phenol to give pyrocatechol and hydroquinone by reaction of phenol with hydrogen peroxide in the presence of a catalyst, characterized in that the reaction is carried out in the presence of an effective amount of a hydroxyaromaticsulfonic acid corresponding to the following formula:

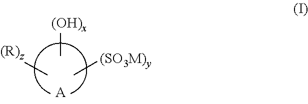

in said formula:
A symbolizing a benzene or naphthalene ring, it being possible for said ring to carry one or more identical or different substituents R,
M representing a hydrogen atom and/or a cation of a metal element from Group Ia of the Periodic Table or an ammonium cation,
x being equal to 1, 2 or 3, preferably 1 or 2,
y being equal to 1 or 2,
z being a number from 0 to 4, preferably 0, 1 or 2,
   and in that the amount of hydrogen peroxide, expressed by the hydrogen peroxide/phenol molar ratio, is less than 0.1, preferably between 0.01 and 0.09.

It has been found, unexpectedly, that the use during the hydroxylation of phenol with hydrogen peroxide of a hydroxyaromaticsulfonic acid corresponding to the formula (I) exerts an effect on the selectivity with regard to the formation of pyrocatechol, the production of this compound being increased with respect to the hydroquinone.

It has been found, surprisingly, that the use of a hydroxyaromaticsulfonic acid corresponding to the formula (I) makes it possible to reduce the amount of hydrogen peroxide introduced while retaining good reaction yields.

In the account which follows the invention, the expression “hydroxyaromaticsulfonic acid” also denotes, for reasons of simplicity, the salts (M other than H).

A hydroxyaromaticsulfonic acid is involved in the process of the invention which corresponds to the general formula (I) in which the residue A, which represents a benzene or naphthalene ring, can carry one or more substituents on the aromatic ring system.

Examples of substituents R are given below but this list does not exhibit a limiting nature. Any substituent can be present on the ring insofar as it does not interfere with the desired product. R represents in particular an alkyl, alkoxy, cycloalkyl, aryl or aralkyl group, an amino or substituted amino group, a nitro group, a nitrile group, a carboxamide group, a carboxyl group or an ester group, preferably an alkyl or aryl ester group.

In the formula (I) M represents a hydrogen atom and/or a cation of a metal element from Group Ia of the Periodic Table, namely lithium, sodium, potassium, rubidium and cesium, or an ammonium cation.

In the present text, reference will be made to the Periodic Table of the Elements published in the Bulletin de la Société Chimique de France, No. 1 (1966).

M is preferably a hydrogen atom, sodium or potassium.

Particular preference is given, among the hydroxyaromaticsulfonic acids of formula (I), to those which correspond to the following formula:

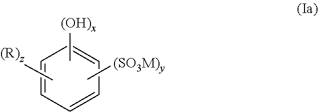

in said formula:
x being equal to 1, 2 or 3, preferably 1 or 2,
y being equal to 1 or 2,
z being a number from 0 to 4, preferably equal to 0, 1 or 2,
M representing a hydrogen atom, sodium or potassium,
R representing an alkyl or alkoxy group having from 1 to 4 carbon atoms or a carboxyl group.

Mention may more particularly be made, among the acids suitable for the process of the invention, of hydroxybenzenesulfonic acids, sulfonated hydroxybenzoic acids, hydroxybenzenedisulfonic acids, dihydroxybenzenedisulfonic acids, hydroxytoluenesulfonic acids, hydroxynaphthalenesulfonic acids and hydroxynaphthalene disulfonic acids, and their mixtures.

Use will preferably be made, among hydroxybenzenesulfonic acids, of 4-hydroxybenzenesulfonic acid, 2-hydroxybenzenesulfonic acid, 5-sulfosalicyclic acid or their mixture. Use may also be made of a hydroxyaromatic acid resulting from the sulfonation of phenol.

Mention may be made, as preferred examples of dihydroxybenzenesulfonic acids employed, of the hydroxyaromaticsulfonic acids resulting from the sulfonation of hydroquinone (1,4-dihydroxybenzene), pyrocatechol (1,2-dihydroxybenzene) and resorcinol (1,3-dihydroxybenzene).

The preferred dihydroxybenzenedisulfonic acids are 5,6-dihydroxy-1,3-benzenedisulfonic acid, 4,6-dihydroxy-1,3-benzenedisulfonic acid or 2,5-dihydroxy-1,4-benzenedisulfonic acid.

The hydroxyaromaticsulfonic acids are available in the solid or liquid form or in the form of an aqueous solution, the concentration of which can vary between 5 and 95% by weight, preferably between 50 and 70% by weight.

The amount of hydroxyaromaticsulfonic acid employed, expressed by the ratio of the number of equivalents of protons (corresponding to the sulfonic functional group) to the number of moles of hydrogen peroxide, can vary as a function of the reaction conditions, in particular the temperature. Thus, said H⁺/H₂O₂ molar ratio can vary between 1×10⁻⁴ and 0.03.

A preferred alternative form of the process of the invention consists in choosing a H⁺/H₂O₂ molar ratio of between 1×10⁻³ and 0.02.

The hydrogen peroxide employed according to the invention can be in the form of an aqueous solution or of an organic solution.

As aqueous solutions are more readily available commercially, they are preferably used.

The concentration of the aqueous hydrogen peroxide solution, although not critical per se, is chosen so as to introduce the least possible amount of water into the reaction medium. Use is generally made of an aqueous hydrogen peroxide solution comprising at least 20% by weight of H₂O₂ and preferably approximately 70%.

The amount of hydrogen peroxide, expressed by the hydrogen peroxide/phenol molar ratio, is less than 0.1, preferably between 0.01 and 0.09 and more preferably between 0.02 and 0.08.

As the amount of water influences the speed of the reaction, it is preferable to minimize its presence, it being possible for the water to be introduced into the reaction medium in particular via the reactants employed.

It is advisable to preferably choose an initial water content of the medium of less than 20% by weight and preferably of less than 10% by weight.

The contents by weight indicated are expressed with respect to the phenol/hydrogen peroxide/water mixture.

This initial water corresponds to the water introduced with the reactants and in particular with the hydrogen peroxide.

An alternative form of the process of the invention consists in adding an agent which complexes the metal ions present in the medium as the latter are harmful to the satisfactory progression of the process of the invention, in particular in the case of phenols where the yields of hydroxylation products are low. Consequently, it is preferable to inhibit the action of the metal ions.

The metal ions harmful to the progression of the hydroxylation are ions of transition metals and more particularly iron, copper, chromium, cobalt, manganese and vanadium ions.

The metal ions are introduced by the reactants and in particular the starting materials and the equipment used. In order to inhibit the action of these metal ions, it is sufficient to carry out the reaction in the presence of one or more complexing agents which are stable with regard to hydrogen peroxide and which give complexes which cannot be decomposed by the strong acids present and in which the metal can no longer exert the chemical activity.

Recourse may in particular be had, as nonlimiting examples of complexing agents, to the various phosphoric acids, such as, for example, orthophosphoric acid, metaphosphoric acid, pyrophosphoric acid or polyphosphoric acids or phosphonic acids, such as 1-hydroxyethylidenediphosphonic acid, phosphonic acid, ethylphosphonic acid or phenylphosphonic acid.

It is also possible to employ the esters of the abovementioned acids and mention may more particularly be made of mono- or dialkyl, mono- or dicycloalkyl or mono- or dialkylaryl orthophosphates, for example ethyl or diethyl phosphate, hexyl phosphate, cyclohexyl phosphate or benzyl phosphate.

The amount of complexing agent depends on the content of metal ions in the reaction medium.

There is obviously no upper limit, it being possible for the amount of complexing agents present to be far in excess with respect to that necessary to complex the metal ions. Generally, an amount representing from 0.01 to 1% by weight of the reaction medium is highly suitable.

In accordance with the process of the invention, the hydroxylation of phenol is carried out at a temperature which can be between 45° C. and 140° C.

A preferred alternative form of the process of the invention consists in choosing a temperature between 60° C. and 120° C.

The reaction is advantageously carried out at atmospheric pressure.

The hydroxylation process is generally carried out without solvent other than that which originates from the reactants, such as the solvent of the hydrogen peroxide.

However, the reaction can also be carried out in a solvent for phenol.

The solvents used must be stable in the presence of hydrogen peroxide.

Mention may be made of nonpolar solvents, such as chlorinated aliphatic hydrocarbons, for example dichloromethane, tetrachloromethane or dichloroethane.

From a practical viewpoint, the process according to the invention is simple to carry out continuously or batchwise.

The catalyst of the invention can be employed in the phenol or in the hydrogen peroxide solution.

Preferably, the following order of the reactants is chosen: the phenol, optionally the complexing agent and the hydroxyaromaticsulfonic acid are introduced.

The reaction medium is brought to the desired temperature and then the hydrogen peroxide solution is added, gradually or continuously.

According to a continuous embodiment, it is possible to send, continuously and in parallel, into one or more reactors in cascade, the phenol, with optionally the complexing agent, the hydrogen peroxide solution; it being possible for the hydroxyaromaticsulfonic acid to be introduced alone or employed in the other reactants.

At the end of the reaction, the unconverted substrate and, if appropriate, the excess hydroxyaromaticsulfonic acid are separated from the hydroxylation products by the usual means, in particular by distillation and/or liquid/liquid extraction, and are returned to the reaction region.

The process for the hydroxylation of phenol under the conditions of the invention makes it possible to obtain a mixture of pyrocatechol and hydroquinone with roughly twice as much pyrocatechol as hydroquinone.

It is particularly advantageous as the yields of diphenols (pyrocatechol+hydroquinone) obtained, expressed by the ratio of the number of moles of diphenols formed (pyrocatechol+hydroquinone) to the number of moles of hydrogen peroxide introduced, are generally at least 70% by weight, preferably between 75 and 87% by weight and more preferably between 80 and 87% by weight.

High yields are obtained while having a relatively low amount of hydrogen peroxide.

Likewise, the amount of catalyst employed is low.

Thus, the invention provides a process capable of being employed on the industrial scale which makes it possible to obtain a pyrocatechol/hydroquinone ratio of between 1.7 and 2.3 and preferably of between 1.9 and 2.2 with a high yield, while resorting to low amounts of hydrogen peroxide and also, preferably, to low amounts of catalyst.

The examples which follow illustrate the invention without, however, limiting it.

In the examples, the following abbreviations signify: The degree of conversion (DC_{H2O2}) of the hydrogen peroxide corresponds to the ratio of the number of moles of hydrogen peroxide converted to the number of moles of hydrogen peroxide introduced.

The yield of diphenols (RY_{diphenols}) corresponds to the ratio of the number of moles of diphenols formed (pyrocatechol+hydroquinone) to the number of moles of hydrogen peroxide introduced.

The pyrocatechol yield (RY_{pyrocatechol}) corresponds to the ratio of the number of moles of pyrocatechol formed to the number of moles of hydrogen peroxide introduced.

The hydroquinone yield (RY_{hydroquinone}) corresponds to the ratio of the number of moles of hydroquinone formed to the number of moles of hydrogen peroxide introduced.

The selectivity for diphenols (S_{diphenols}) corresponds to the ratio of the number of moles of diphenols formed (pyrocatechol+hydroquinone) to the number of moles of hydrogen peroxide converted.

The PC/HQ ratio is defined by the ratio of the number of moles of pyrocatechol to the number of moles of hydroquinone.

### EXAMPLES

The procedure which will be followed in all the examples is given below.

The following are charged at 50° C. to a 250 ml jacketed reactor equipped with a stirring system of 4 inclined blades type, with a vertical reflux condenser, with a nitrogen inlet and with a heating device:
117.6 g (1.25 mol) of phenol,
a catalyst in a proportion generally of 700 molar ppm with respect to the phenol, the nature of which is specified in the summarizing tables.

The mixture is brought to a temperature of 80° C. under a nitrogen atmosphere and then 3.03 g of 70% by weight hydrogen peroxide (i.e. 0.0625 mol of hydrogen peroxide) are added over 30 min using a syringe driver. An increase in temperature accompanied by a coloring of the reaction mixture are generally observed.

Subsequently, the mixture is heated to 90° C.

At the end of the reaction, the reaction mixture is cooled to 50° C. and the diphenols formed are quantitatively determined by high performance liquid chromatography.

The specific conditions and results are collated in summarizing tables.

### Examples 1 to 3

In these examples, use is made of a catalyst according to the invention, namely a hydroxyaromaticsulfonic acid.

The conditions and results obtained are recorded in table (I).

TABLE (I)

| Example | | | |
|---|---|---|---|
| reference | 1 | 2 | 3 |
| Catalyst | 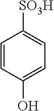 | 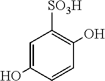 | 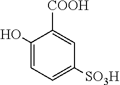 |
| mol % | 1.5 | 1.4 | 1.4 |
| cata./H₂O₂ | | | |
| weight % of | 0.8 | 0.8 | 0.8 |
| water | | | |
| H₂O₂/phenol % | 5 | 5 | 5 |
| RY HQ | 26 | 23 | 28 |
| RY PC | 56 | 48 | 57 |
| RY (HQ + | 82 | 71 | 85 |
| PC) | | | |
| PC/HQ ratio | 2.2 | 1.9 | 2.1 |
| DC H₂O₂ | 98 (1 h) | 97 (1 h) | 99 (1h) |
| S (PC + | 83 | 73 | 86 |
| HQ)/H₂O₂ | | | |

### Example 4

The phenol (with the complexing agent, orthophosphoric acid, in a proportion of 0.025% of the weight of the phenol), the hydrogen peroxide and the catalyst are introduced, in parallel and continuously, into a cascade of 500 ml glass reactors.

Each jacketed reactor is equipped with a mechanical stirring system of 4 inclined blades type, with a system for regulating the temperature, with a vertical reflux condenser and with a nitrogen inlet.

500 g/h (5.32 mol) of phenol, 14.4 gill of 70% by weight hydrogen peroxide (i.e. 0.30 mol/h) and 5-sulfo-salicylic acid dihydrate (0.96 g/h, i.e. approximately 700 molar ppm with respect to the phenol) are charged using pumps.

The temperature profile is as follows: 85° C. for the 1^{st} reactor, 92° C. for the second and 95° C. for the third.

After a stabilization time (approximately 1 h), the diphenols formed are quantitatively determined by high performance liquid chromatography and the hydrogen peroxide is quantitatively determined by potentiometry.

The operating conditions and results obtained in the 3^{rd} reactor are recorded in table (II).

TABLE (II)

| Example reference | 4 |
|---|---|
| Catalyst | 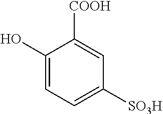 |
| mol % cata./H₂O₂ | 1.25 |
| weight % of water | 0.8 |
| RY HQ | 24 |
| H₂O₂/phenol % | 5.6 |
| RY PC | 49.5 |
| RY (HQ + PC) | 73.5 |
| PC/HQ ratio | 2.1 |
| DC H₂O₂ | 91 |
| S (PC + HQ)/H₂O₂ | 81 |

### Example 5

The phenol (with the complexing agent, orthophosphoric acid, in a proportion of 0.025% of the weight of the phenol), the hydrogen peroxide and the catalyst are introduced, in parallel and continuously, into a cascade of 500 ml glass reactors.

Each jacketed reactor is equipped with a mechanical stirring system of 4 inclined blades type, with a system for regulating the temperature, with a vertical reflux condenser and with a nitrogen inlet.

517 g/h (5.49 mol) of phenol, 9.5 g/h of 70% by weight hydrogen peroxide (i.e. 0.195 mol/h) and 4-phenylsulfonic acid as a 65% by weight aqueous solution (1.03 g/h, i.e. 700 molar ppm with respect to the phenol) are charged using pumps.

The temperature profile is as follows: 89° C. for the 1 reactor, 90° C. for the second and 90° C. for the third.

After a stabilization time (approximately 1 h), the diphenols formed are quantitatively determined by high performance liquid chromatography and the hydrogen peroxide is quantitatively determined by potentiometry.

The operating conditions and results obtained in the 3^{rd} reactor are recorded in table (III).

TABLE (III)

| Example reference | 5 |
|---|---|
| Catalyst | 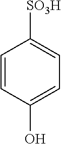 |
| mol % cata./H₂O₂ | 1.95 |
| weight % of water | 0.6 |
| H₂O₂/phenol % | 3.55 |
| RY HQ | 26 |
| RY PC | 51 |
| RY (HQ + PC) | 77 |
| PC/HQ ratio | 2.0 |
| DC H₂O₂ | 92 |
| S (PC + HQ)/H₂O₂ | 83 |

### Comparative Tests A to E

In these examples, use is made of catalysts of sulfonic acid type but which are not hydroxylated aromatic sulfonic compounds.

The conditions and results are recorded in table (IV).

TABLE (IV)

| Test reference | A | B | C | D | E |
|---|---|---|---|---|---|
| Catalyst | 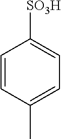 | 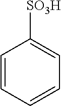 | 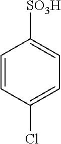 | 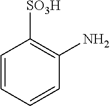 | 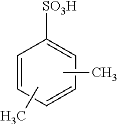 |
| mol % | 0.5 | 1.4 | 1.4 | 1.3 | 1.5 |
| cata./H₂O₂ | | | | | |
| weight % of | 0.8 | 0.8 | 0.8 | 0.8 | 0.8 |
| water | | | | | |
| H₂O₂/phenol % | 5 | 5 | 5 | 5 | 5 |
| RY HQ | 23 | 25 | 21 | 13 | 4 |
| RY PC | 48 | 53 | 51 | 38 | 23 |
| RY (HQ + PC) | 71 | 78 | 72 | 51 | 27 |
| PC/HQ ratio | 2.1 | 2.2 | 2.5 | 3 | 5.9 |
| DC H₂O₂ | 92 (1 h) | 99 (1 h) | 100 | 22 (1 h) | 99 (1 h) |
| | | | (1 h) | 99 (5 h) | |
| S (PC + HQ)/H₂O₂ | 77 | 78 | 72 | 52 | 27 |

1-13. (canceled)

14. A process for the hydroxylation of phenol to yield pyrocatechol and hydroquinone comprising:
reacting the phenol with hydrogen peroxide in the presence of a catalyst and an effective amount of a hydroxyaromaticsulfonic acid of formula: 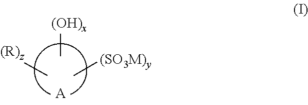
wherein:
A represents a benzene or naphthalene ring, optionally carrying one or more identical or different substituents, R,
M represents a hydrogen atom and/or a cation comprising an alkali metal cation or an ammonium cation,
x is equal to 1, 2, or 3,
y is equal to 1 or 2, and
z is a number from 0 to 4;
further wherein the yield ratio of pyrocatechol to hydroquinone ranges from 1.7 to 2.3.

15. A process for the hydroxylation of phenol to yield pyrocatechol and hydroquinone comprising:
reacting the phenol with hydrogen peroxide in the presence of a catalyst and an effective amount of a hydroxyaromaticsulfonic acid of formula: 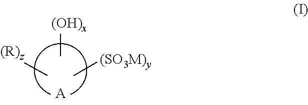
wherein:
A represents a benzene or naphthalene ring, optionally carrying one or more identical or different substituents, R,
M represents a hydrogen atom and/or a cation comprising an alkali metal cation or an ammonium cation,
x is equal to 1, 2, or 3,
y is equal to 1 or 2, and
z is a number from 0 to 4;
further wherein the molar ratio of hydrogen peroxide to phenol is less than 0.1.

16. The process of claim 14, wherein R represents an alkyl, alkoxy, cycloalkyl, aryl or aralkyl group; an amino or substituted amino group; a nitro group; a nitrile group; a carboxamide group; a carboxyl group; or an ester group.

17. The process of claim 15, wherein R represents an alkyl, alkoxy, cycloalkyl, aryl or aralkyl group; an amino or substituted amino group; a nitro group; a nitrile group; a carboxamide group; a carboxyl group; or an ester group.

18. The process of claim 14, wherein the hydroxyaromaticsulfonic acid comprises a compound of formula (Ia): 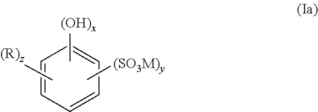
wherein:
x is equal to 1, 2, or 3,
y is equal to 1 or 2, and
z is a number from 0 to 4;
M represents a hydrogen atom, sodium, or potassium, and
R represents an alkyl or alkoxy group having from 1 to 4 carbon atoms; or a carboxyl group.

19. The process of one of claim 15, wherein the hydroxyaromaticsulfonic acid comprises a compound of formula (Ia): 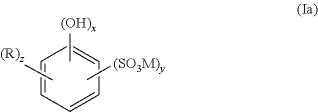
wherein:
x is equal to 1, 2, or 3,
y is equal to 1 or 2, and
z is a number from 0 to 4;
M represents a hydrogen atom, sodium, or potassium, and
R represents an alkyl or alkoxy group having from 1 to 4 carbon atoms; or a carboxyl group.

20. The process of claim 14, wherein the hydroxyaromaticsulfonic acid comprises a hydroxybenzenesulfonic acid, a sulfonated hydroxybenzoic acid, a hydroxybenzenedisulfonic acid, a dihydroxybenzenedisulfonic acid, a hydroxytoluenesulfonic acid, a hydroxynaphthalenesulfonic acid, a hydroxynaphthalenedisulfonic acid, or a mixture thereof.

21. The process of claim 15, wherein the hydroxyaromaticsulfonic acid comprises a hydroxybenzenesulfonic acid, a sulfonated hydroxybenzoic acid, a hydroxybenzenedisulfonic acid, a dihydroxybenzenedisulfonic acid, a hydroxytoluenesulfonic acid, a hydroxynaphthalenesulfonic acid, a hydroxynaphthalenedisulfonic acid, or a mixture thereof.

22. The process of claim 20, wherein:
the hydroxybenzenesulfonic acid comprises 4-hydroxybenzenesulfonic acid, 2-hydroxybenzenesulfonic acid, 5-sulfosalicyclic acid, or a mixture thereof;
the dihydroxybenzenesulfonic acid comprises a sulfonic acid resulting from the sulfonation of hydroquinone, pyrocatechol or resorcinol; and
the dihydroxybenzenedisulfonic acid comprises 5,6-dihydroxy-1,3-benzenedisulfonic acid, 4,6-dihydroxy-1,3-benzenedisulfonic acid, or 2,5-dihydroxy-1,4-benzenedisulfonic acid.

23. The process of claim 21, wherein:
the hydroxybenzenesulfonic acid comprises 4-hydroxybenzenesulfonic acid, 2-hydroxybenzenesulfonic acid, 5-sulfosalicyclic acid, or a mixture thereof;
the dihydroxybenzenesulfonic acid comprises a sulfonic acid resulting from the sulfonation of hydroquinone, pyrocatechol or resorcinol; and
the dihydroxybenzenedisulfonic acid comprises 5,6-dihydroxy-1,3-benzenedisulfonic acid, 4,6-dihydroxy-1,3-benzenedisulfonic acid, or 2,5-dihydroxy-1,4-benzenedisulfonic acid.

24. The process of claim 14, wherein the amount of hydroxyaromaticsulfonic acid, expressed by the H⁺/H₂O₂ molar ratio, ranges from 1×10⁻⁴ to 0.03.

25. The process of claim 24, wherein the amount of hydroxyaromaticsulfonic acid, expressed by the H⁺/H₂O₂ molar ratio, ranges from 1×10⁻³ to 0.02.

26. The process of claim 14, wherein the hydrogen peroxide/phenol molar ratio ranges from 0.01 to 0.09.

27. The process of claim 26, wherein the hydrogen peroxide/phenol molar ratio ranges from 0.02 to 0.08.

28. The process of claim 14, wherein said process is carried out in the presence of at least one agent that complexes transition metal ions and that is stable under the reaction conditions.

29. The process of claim 28, wherein said agent comprises a phosphoric acid and/or a phosphonic acid.

30. The process of claim 29, wherein:
the phosphoric acid comprises orthophosphoric acid, metaphosphoric acid, pyrophosphoric acid, or polyphosphoric acid, and
the phosphonic acid comprises 1-hydroxyethylidenediphosphonic acid, ethylphosphonic acid, phenylphosphonic acid, or their acid esters.

31. The process of claim 14, wherein the reaction is carried out at a temperature ranging from 45° C. to 140° C.

32. The process of claim 14, comprising:
introducing the phenol, the hydroxyaromaticsulfonic acid, and optionally a complexing agent,
bringing the reaction medium to a reaction temperature, and
gradually and/or continuously adding the hydrogen peroxide in the form of a solution to the reaction mixture.

33. The process of claim 14, wherein the process is carried out batchwise.

34. The process of claim 14, wherein the process is carried out continuously.

35. The process of claim 14, wherein the pyrocatechol/hydroquinone ratio ranges from 1.9 to 2.2.
